(19) United States
(12) Patent Application Publication
(10) Pub. No.: US 2026/0232900 A1
(43) Pub. Date: Aug. 13, 2026

(54) INTRAVENOUS LINE STRAP WITH DISLODGEMENT PROTECTION

(71) Applicant: CareFusion 303, Inc., San Diego, CA (US)

(72) Inventors: Maurya Sundar Vijaya Kumar, Limerick (IE); John Meaney, Limerick (IE); Warren Greer, Limerick (IE); Stephen Murphy, Limerick (IE)

(21) Appl. No.: 19/051,053

(22) Filed: Feb. 11, 2025

(51) Int. Cl. A61M 5/168

(52) U.S. Cl. CPC A61M 5/16813; A61M 5/16854; A61M 2005/16863; A61M 2207/00

(57) ABSTRACT

An intravenous (IV) cartridge assembly can reduce or prevent dislodgement of IV lines from patients receiving fluids via IV administration. The IV cartridge assembly includes an anchor that couples the IV cartridge assembly to patients, an internal channel configured to receive the IV line, a roller wheel that rolls on top of the IV line along a tapered track in the IV cartridge assembly, and a protrusion in the internal channel that raises the IV line into the path of the roller wheel. In its default position, the roller wheel does not interfere with fluid flow through the IV line. However, when a large enough force is applied to the IV line, the roller wheel rolls along the IV line towards the protrusion and pinches the IV line against the protrusion to occlude the IV line. This occlusion can trigger an alarm calls a caregiver to reset the IV line.

### TECHNICAL FIELD

The present disclosure relates generally to intravenous infusion pump mechanisms. In particular, the present disclosure relates to mechanisms that limit intravenous line dislodgement and the ensuing consequences.

### BACKGROUND

In the medical field, infusion pumps are often used to administer precise amounts of infusates from an external bag to a patient via an intravenous (IV) administration set. The IV administration typically includes a plastic or flexible tubing with connectors on either end—with one end attached to a fluid source such as a saline bag or medication vial, while the other end is connected to a needle or catheter inserted into a vein. As the pump compresses and releases the tubing of the pumping segment in a rhythmic motion, it generates a pulsatile flow, effectively propelling fluid through the tubing of the IV administration set and into the patient's bloodstream.

### SUMMARY

While IV administration of fluids (medication, blood, saline, etc.) to patients is a widely used method of fluid delivery, IV administration is subject to periodic failure before treatment is complete due to dislodgement of the IV cannula from the patient. This can be because the IV line often ends up getting pulled by heavily medicated patients who are confused and want to remove the IV line, young patients that are fidgeting with the IV line, etc. The IV line can also get dislodged by accident when the IV line catches on a person or equipment in the vicinity. When the IV line dislodges, fluid spills out of the IV line, which not only makes a mess that caregivers have to spend time cleaning, but also wastes fluid. Further, a caregiver rarely knows when the IV line became dislodged or how much fluid was lost in the spill. In the case of an IV administration of a medication, the caregiver may not know how much medication was delivered to the patient. Consequently, the patient may lose out on medication, which negatively impacts treatment.

Accordingly, it is desirable to have a device, such as the IV cartridge assembly described herein, that can be incorporated into an IV set that reduces or prevents the loss of medication on the occurrence of a dislodgement of the IV cannula from the patient. The IV cartridge assembly can occlude the IV line and alert the caregiver when the patient is trying to remove the IV line from his arm or when a force is otherwise pulling the IV line out of the patient's arm. Moreover, the IV cartridge assembly can quickly and conveniently be reset by the caregiver for repeated use.

The present disclosure addresses the aforenoted problems by providing an IV cartridge assembly for mitigating the consequences of IV line dislodgement by occluding the IV line upon and alerting caregivers when the IV line is pulled. According to various implementations, the disclosed IV cartridge assembly comprises an anchor configured to couple the IV cartridge assembly to a patient; an internal channel traversing the IV cartridge assembly, the internal channel being configured to receive an IV line; a protrusion formed in a bed of the internal channel; a tapered track within the IV cartridge assembly and above the internal channel; and a roller wheel configured to roll along the tapered track, wherein the roller wheel is configured to sit on top of the IV line without interfering with fluid flow through the IV line, wherein, in response to the IV line being pulled, the roller wheel is configured to roll towards the protrusion, wherein, when the roller wheel reaches the protrusion, the roller wheel is configured to pinch the IV line against the protrusion and occlude the IV line. Other aspects include corresponding systems and methods for implementation of the corresponding apparatus and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts a perspective view of a first implementation of the IV cartridge coupled to an IV line, according to various aspects of the subject technology.

FIG. 2A depicts a cross-sectional view of the first implementation of the IV cartridge coupled to the IV line, with the roller wheel in the default position, according to various aspects of the subject technology.

FIG. 2B depicts a cross-sectional view of the first implementation of the IV cartridge coupled to the IV line, with the roller wheel in the occluded position, according to various aspects of the subject technology.

FIG. 3 depicts a perspective view of the first implementation of the IV cartridge assembly coupled to the IV line and in the opened configuration, according to various aspects of the subject technology.

FIG. 4 depicts a perspective view of a second implementation of the IV cartridge assembly coupled to an IV line, according to various aspects of the subject technology.

FIG. 5 depicts a flow chart of the method of manufacture of the IV cartridge assembly, according to various aspects of the subject technology.

### DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

As will be described further, the subject technology includes an IV cartridge assembly that is configured to be positioned along an IV line. The IV cartridge assembly includes an internal channel that can receive the IV line and a tapered track that receives a roller wheel. When a force pulls the IV line through the internal channel of the IV cartridge assembly, the roller wheel rolls on top of the IV line. If the force is large enough, the roller wheel will roll until it reaches a protrusion in the internal channel beneath the IV line. When the roller wheel reaches the protrusion, the roller wheel and the protrusion pinch the IV line to partially or fully occlude the IV line to interrupt or stop fluid flow. The IV cartridge assembly can be reset and reused by manually rolling the roller wheel away from the protrusion, back to its original position along the tapered track.

In some implementations, the roller wheel is concealed from the surrounding environment to prevent a patient or other person from accidentally or intentionally tampering with the IV cartridge assembly. For example, the IV cartridge assembly can comprise a top portion connected to a bottom portion by a hinge. When in use, the IV cartridge assembly is closed and the internal components and features of the IV cartridge assembly are concealed from the patient and the surrounding environment. When the IV cartridge assembly needs to be reset, the top portion can rotate away from the bottom portion via the hinge, and a caregiver can access the roller wheel to roll it back to the original, default position.

Some of the advantages of embodiments consistent with the present disclosure include simple and cost-effective manufacturing processes and easy adaptation to IV lines of various sizes and materials. Further, the IV cartridge assembly described herein is reusable and can easily be reset, which reduces the duration of the fluid flow interruption caused by the occlusion (e.g., by about one to fifteen minutes, depending on the specific infusion setup). Additionally, the IV cartridge assembly can be reset while still coupled to the patient. This gives the caregiver a free hand without requiring the caregiver to place the IV cartridge assembly on a different surface, which would require the caregiver to sanitize the IV cartridge assembly every time she resets it.

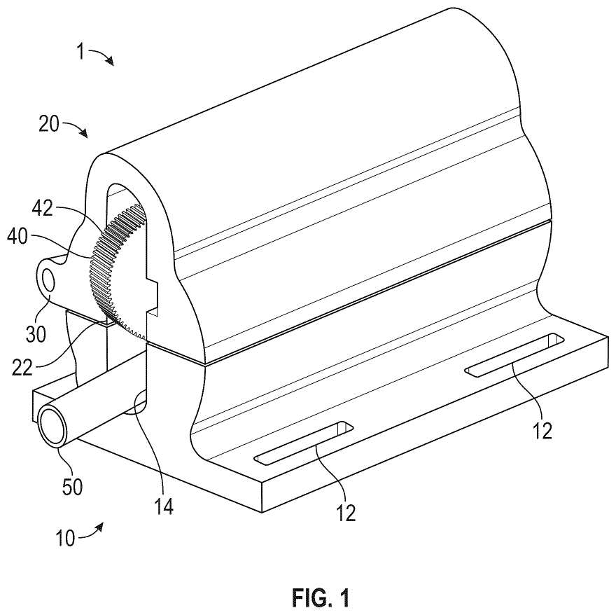

FIG. 1 depicts a perspective view of a first implementation of the IV cartridge assembly coupled to an IV line, according to various aspects of the subject technology. The IV cartridge assembly 1 (which can also be referred to as the IV cartridge 1 or the cartridge assembly 1) is configured to be coupled to a patient and to receive an IV set (at the IV line 50). Specifically, the IV cartridge assembly 1 has anchors 12 by which the IV cartridge assembly 1 is coupled to a patient (e.g., at the patient's arm). In the implementation depicted in FIG. 1, the anchors 12 are slots that can receive straps to tie, strap, fasten (e.g., via hook and loop fasteners), or otherwise couple the IV cartridge assembly 1 to the patient. In other implementations, the anchors 12 can include a singular slot, one or more apertures, adhesives, straps, fasteners, etc.

The IV cartridge assembly 1 comprises a base portion 10 and a top portion 20 movably coupled together by a hinge 30. As will be described further, the top portion 20 may be opened to insert an IV line 50 of the IV set within an internal channel 14 formed within the IV cartridge assembly 1. According to various implementations, the internal channel 14 forms a bed for the IV line 50 along a bottom of the assembly 1, and extending through the entirety of the IV cartridge assembly 1, such that the IV line 50 can enter the IV cartridge assembly 1 through a first end of the IV cartridge assembly 1 and exit through a second end. The internal channel 14 is sized to receive different types of IV lines 50. For example, the internal channel 14 can have a wide bed (i.e., a wide base or basin) that can accommodate both large and small IV lines 50. Alternatively, different implementations of the IV cartridge assembly 1 can have internal channels 14 with differently sized beds, such that different implementations of the IV cartridge assembly 1 are compatible with different sizes of IV lines 50.

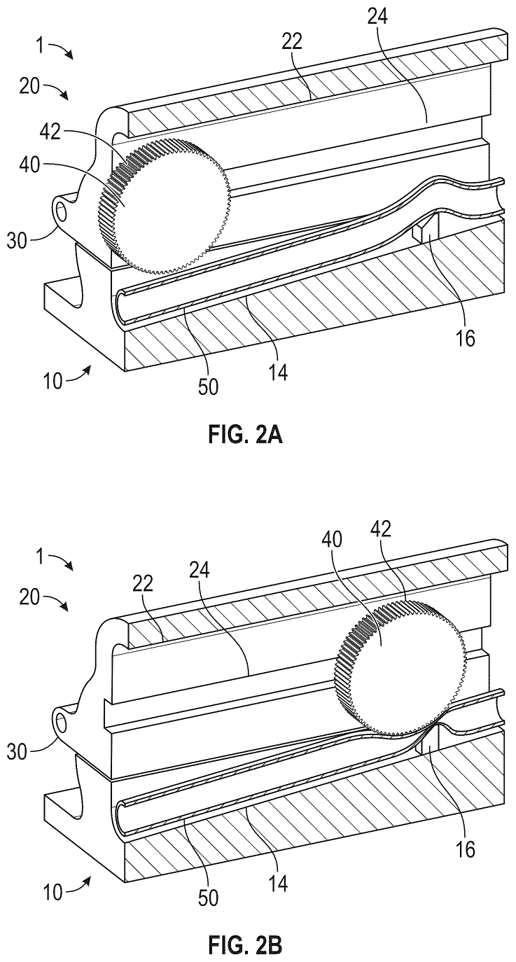

A tapered track 22, described in greater detail with regard to FIGS. 2A and 2B, is formed within the IV cartridge assembly 1 above the internal channel 14. A roller wheel 40 is movably coupled to the tapered track 22. As depicted in FIG. 1, the roller wheel 40 is a cylindrical disc. In other implementations of the IV cartridge assembly 1, the roller wheel 40 can be a ring or a wheel (e.g., with spokes and/or a hub). In implementations where the roller wheel 40 is a wheel with a hub, the tapered track 22 can have an additional slot, such as hub slot 24. The hub of the roller wheel 40 fits inside the hub slot 24 and rolls along the hub slot 24 when the roller wheel 40 rolls along the tapered track 22 and the IV line 50.

The roller wheel 40 is configured to sit on top of the IV line 50 when the IV line 50 is in the internal channel 14 (see FIG. 2A). In this configuration (which can be referred to as the default position), the roller wheel 40 does not prevent or interfere with fluid flow through the IV line 50. The roller wheel 40 is also configured to roll on top of the IV line 50 along the tapered track 22 when a force is applied to the IV line 50 (e.g., by a patient or other person pulling on the IV line 50, either intentionally or accidentally).

In particular, when a force is applied to the IV line 50, the IV line 50 slides through the internal channel 14 in the direction of the force while the roller wheel 40 rolls along the tapered track 22 on top of the IV line 50 in a direction opposite the force. The roller wheel 40 rolls on top of the IV line 50 towards a protrusion (e.g., protrusion 16 depicted in FIGS. 2A and 2B) in the internal channel 14. The roller wheel 40 does not interrupt fluid flow through the IV line 50 as the roller wheel 40 rolls along the tapered track 22 until the roller wheel 40 reaches the protrusion. When the roller wheel 40 reaches the protrusion, the IV line 50 is occluded (i.e., pinched) between the roller wheel 40 and the protrusion (see FIG. 2B). In this occluded configuration, the roller wheel 40 reduces fluid flow through the IV line 50 by pinching the IV line 50 against the protrusion to reduce the area of the cross-section of the IV line 50 at the pinch point. Optionally, the roller wheel 40 completely stops fluid flow through the IV line 50 by pinching the IV line 50 shut against the protrusion.

In some implementations, the IV cartridge assembly 1 may be configured to couple with one or more pressure sensors (e.g., as part of an infusion pump). The pressure sensors, or pressure transducers, measure the pressure in the IV line 50 to identify when fluid flow through the IV line 50 is being interrupted (e.g., when the roller wheel 40 pinches the IV line 50 against the protrusion in the internal channel 14, as described above). In some implementations, the IV cartridge assembly is coupled with a single pressure sensor that measures the pressure in one part of the IV line 50. In these implementations, a full or partial occlusion in the IV line 50 is indicated by a change in the pressure measurement. For example, if the pressure measurement suddenly decreases, this is an indication that fluid flow through the IV line 50 has slowed down or entirely stopped. In some implementations, the IV set includes two pressure sensors (e.g., one on each side of the protrusion in the internal channel 14 of the IV cartridge assembly 1 or one on each side of the IV cartridge assembly 1). In these implementations, the two pressure sensors will have the same (or at least a similar) pressure measurement when fluid is flowing through the IV line 50. When the difference between the pressure measurements obtained by the two pressure sensors increases, this means that the IV line 50 is at least partially occluded.

Further, the IV cartridge assembly 1 and the one or more pressure sensors may be configured to couple with a processor. In such implementations, the processor analyzes the pressure measurements obtained by the pressure sensors and notifies a caregiver (e.g., by setting off an alarm or a signal) when the change in pressure in the IV line 50 exceeds a predetermined threshold. Immediately notifying the caregiver can reduce the length of interruptions in fluid delivery and limit the amount of fluid that leaks out of the IV line 50. Additionally, the pressure difference threshold can be increased or decreased to change when the caregiver is notified. For example, for patients in critical condition for which interruptions in fluid delivery are life-threatening, the threshold for the pressure difference can be decreased so that the caregiver is notified even when the IV line 50 is only partially occluded, which can occur when the roller wheel 40 is rolling towards the protrusion 16 but has not yet reached the occluded position. On the other hand, in situations where fluid delivery is less critical (e.g., where saline is being delivered to the patient to keep a vein open to make future medication delivery more comfortable), the threshold for the pressure difference can be increased so that the caregiver is notified only when the IV line 50 is already occluded.

FIG. 2A depicts a cross-sectional view of the first implementation of the IV cartridge coupled to the IV line, with the roller wheel in the default position, according to various aspects of the subject technology. As described above with respect to FIG. 1, the internal channel 14 is formed within the IV cartridge assembly 1 and is configured to receive the IV line 50. In the implementation shown, the internal channel 14 is positioned towards the bottom of the IV cartridge assembly 1 (i.e., near the base portion of the IV cartridge assembly 1 and the anchors (anchors 12, depicted in FIG. 1)), such that the IV line 50 is only slightly raised above the patient's arm when the IV line 50 is coupled to the IV cartridge assembly 1. Minimizing the distance between the internal channel 14 and the patient arm—and, thus, minimizing the elevation of the IV line 50 relative to the patient's arm—facilitates consistent fluid flow through the IV line 50 and the IV cartridge assembly 1.

FIG. 2A also depicts the roller wheel 40 resting on top of the IV line 50. As shown, in this default position, the roller wheel 40 does not close (or otherwise reduce the cross-sectional area of) the IV line 50, so fluid flow can continue through the IV line 50. FIG. 2A depicts that the circumference of the roller wheel 40 (i.e., the curved surface of the cylindrical disc that constitutes the roller wheel 40) has ridges 42. These ridges 42 create friction between the roller wheel 40 and the IV line 50. Not only does this friction resist movement of the IV line 50 through the IV cartridge assembly 1 when the force applied to the IV line 50 is not enough to overcome the static friction between the roller wheel 40 and the IV line 50, but this friction also helps the roller wheel 40 roll up the IV line 50 when the force applied overcomes the static friction. Specifically, the ridges 42 grip the IV line 50 so that the roller wheel 40 rolls up the IV line 50 instead of rotating in place as the IV line 50 slides through the IV cartridge assembly 1. Different implementations of the roller wheel 40 can have ridges 42 with different sizes and/or quantities, which would change the amount of friction between the roller wheel 40 and the IV line 50. Optionally, the IV cartridge assembly 1 can have a roller wheel 40 with different structures that grip onto the IV line 50. For instance, instead of ridges 42, the roller wheel 40 can have bumps, treads, grooves, etc.

As illustrated in FIG. 2A, the internal channel 14 has a protrusion 16 that extends upwards (i.e., towards the tapered track 22) from the bed of the internal channel 14. When the IV line 50 is inside the internal channel 14, the protrusion 16 slightly raises part of the IV line 50 towards the tapered track 22. Because the protrusion 16 raises the IV line 50 into the path of the roller wheel 40, the roller wheel pinches the IV line 50 against the protrusion 16 when the roller wheel is on top of the protrusion 16 (i.e., in the occluded position). While the implementation of the IV cartridge assembly 1 depicted in FIGS. 2A and 2B has only one protrusion 16, other implementations of the IV cartridge assembly 1 can have multiple protrusions 16. For example, forces can be applied to the IV line 50 on either side of the IV cartridge assembly 1, which means the IV line 50 can be pulled in two directions relative to the IV cartridge assembly 1. To limit or address dislodgements caused by pulling in both directions, the internal channel 14 can have one protrusion 16 near each end of the IV cartridge assembly. This would provide two occluded positions at which the roller wheel 40 can pinch the IV line 50 against a protrusion 16 to occlude the IV line 50 or to trigger an occlusion notification to the caregiver.

FIG. 2B depicts a cross-sectional view of the first implementation of the IV cartridge assembly coupled to the IV line, with the roller wheel in the occluded position, according to various aspects of the subject technology. As explained above with respect to FIG. 1, when the roller wheel is in the occluded position, the roller wheel 40 and the protrusion 16 squeeze the IV line 50, which reduces the cross-sectional area of the IV line 50 and restricts fluid flow through the IV line 50. The roller wheel 40 and the protrusion 16 can also pinch the IV line 50 shut, such that fluid entirely stops flowing through the IV line 50.

The roller wheel 40 transitions between the default position (shown in FIG. 2A) and the occluded position (shown in FIG. 2B) when a force is applied to the IV line 50 that pulls the IV line 50 through the internal channel 14 of the IV cartridge assembly 1. As the force is applied, the IV line 50 moves in the direction of the force, which causes the roller wheel 40 to roll along the tapered track 22 on the IV line 50 in the direction opposite the force (towards the protrusion 16). The roller wheel 40 squeezes the IV line 50 against the protrusion 16 because the tapered track 22 converges with the internal channel 14 at the protrusion 16. Thus, when the force is applied to the IV line 50, the roller wheel 40 moves towards the protrusion 16 in both the horizontal and vertical directions.

A threshold force needs to be applied to the IV line 50 in order for the roller wheel 40 to reach the occluded position. The threshold force can be reached by a frequent or continuous application of a smaller force and/or by application of a singular, larger force. Different implementations of the IV cartridge assembly 1 can have different threshold forces. When the IV line 50 is coupled to an IV cartridge assembly 1 with a higher threshold force, more force is required to move the roller wheel 40 into the occluded position. On the other hand, when the IV line 50 is coupled to an IV cartridge assembly 1 with a lower threshold force, the IV line 50 can be occluded even when a small amount of force is applied because only a small force is required for the roller wheel 40 to transition from the default position to the occluded position. An IV cartridge assembly 1 with a higher threshold force can be useful for patients who frequently pull on the IV line 50 (e.g., children) or for patients for which uninterrupted fluid delivery is critical (e.g., when the fluid is life-saving medication) because less of the IV line 50 has to be pulled through the IV cartridge assembly 1 before the IV line 50 is occluded and a caregiver is notified. If less of the IV line 50 has to be pulled through the IV cartridge assembly before the caregiver is notified to come readjust the IV line 50, then the IV line 50 is less likely to become dislodged from the patient or the IV set.

Two major factors that determine the threshold force are the lengths and slopes of the internal channel 14 and the tapered track 22. The lengths of the internal channel 14 and the tapered track 22 can be adjusted to change the threshold force of the IV cartridge assembly 1. For example, increasing the lengths of the internal channel 14 and the tapered track 22 increases the distances that the roller wheel 40 must travel while transitioning between the default position and the occluded position. Accordingly, a greater threshold force (e.g., pulling on the IV line 50 for a longer period of time) is required to roll the roller wheel 40 towards the protrusion, which means that a longer internal channel 14 and a longer tapered track 22 increases the threshold force required for the IV cartridge assembly 1 to occlude the IV line 50.

Similarly, the slopes of the internal channel 14 and the tapered track 22 can be adjusted to change the threshold force of the IV cartridge assembly 1. While increasing the slope of the internal channel 14 relative to the tapered track 22 (or increasing the slope of the tapered track 22 relative to the internal channel 14) increases the amount of force required to roll the roller wheel 40 towards the protrusion 16, decreasing the slope decreases the amount of force required. In other words, increasing the angle between the internal channel 14 and the tapered track 22 increases the threshold force, while decreasing the angle decreases the threshold force.

In implementations with two protrusions 16, as described above, one or both of the internal channel 14 and the tapered track 22 can curve (either dipping downwards or bowing upwards) at a position between the two protrusions 16. In these implementations, the roller wheel 40 can be equidistant from both protrusions 16 when the roller wheel 40 is in the default position. Said differently, when the roller wheel 40 is in the default position, the IV cartridge assembly 1 is generally symmetrical about the roller wheel 40.

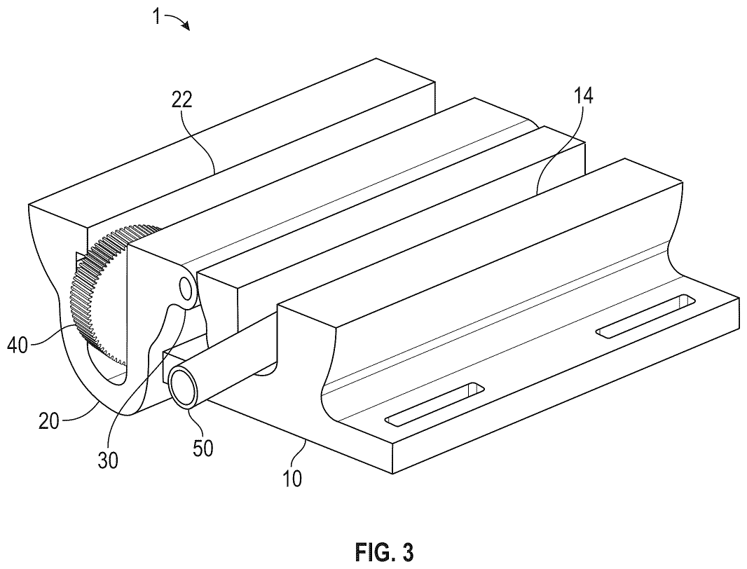

FIG. 3 depicts a perspective view of the first implementation of the IV cartridge assembly coupled to the IV line and in the opened configuration, according to various aspects of the subject technology. In the implementations of the IV cartridge assembly 1 depicted in FIGS. 1-3, the IV cartridge assembly 1 comprises a base portion 10 and a top portion 20 movably coupled together by a hinge 30. The base portion comprises the anchors 12, the internal channel 14, and the protrusion 16, while the top portion comprises the tapered track 22 and conceals the roller wheel 40 from the surrounding environment when the IV cartridge assembly 1 is in the closed configuration (depicted in FIGS. 1-2B). The IV cartridge assembly 1 is designed to be locked in the closed configuration when the IV cartridge assembly 1 is in use.

When the IV cartridge assembly 1 needs to be reset after the IV line 50 has been occluded, a caregiver with a key can unlock the IV cartridge assembly 1 and rotate the top portion 20 away from the base portion 10 via the hinge 30 to access the roller wheel 40. After resetting the roller wheel 40 (i.e., by rolling the roller wheel 40 back into the default position), the caregiver closes the IV cartridge assembly 1 and locks the top portion 20 relative to the base portion 10. Accordingly, the hinge 30 and the lock allow the caregiver to reset the IV cartridge assembly 1 without decoupling the IV cartridge assembly 1 from the patient and from the IV line 50. Because the IV cartridge assembly 1 does not leave the patient's arm, the caregiver can quickly and cleanly reset the IV set without wasting time, losing fluids, or contaminating any part of the IV set.

Moreover, when the IV cartridge assembly 1 is in the closed configuration, the interior of the IV cartridge assembly 1 is concealed and locked away, which prevents interference with the roller wheel 40. For example, the patient (or a third party) cannot roll the roller wheel 40 back and forth in the IV cartridge assembly either by accident or on purpose, which can reduce instances of accidental occlusions and, thus, unnecessary notifications to the caregiver.

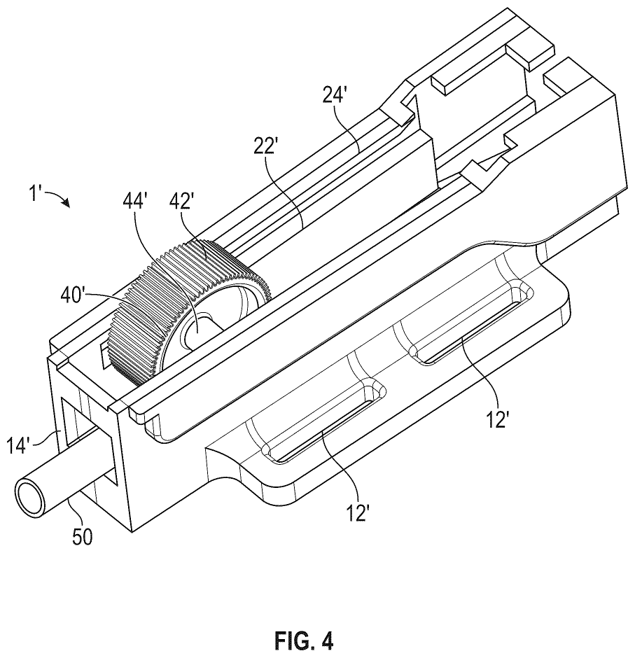

FIG. 4 depicts a perspective view of a second implementation of the IV cartridge assembly coupled to an IV line, according to various aspects of the subject technology. The IV cartridge assembly 1′ of the second implementation may be configured and operable like the IV cartridge assembly 1, except for the differences described below. As depicted in FIG. 4, the IV cartridge assembly 1′ has a roller wheel 40′ that is exposed to the surrounding environment. This allows caregivers to reset the IV cartridge assembly 1′ quickly and conveniently because there is no need to unlock or open the IV cartridge assembly 1′. This implementation of the IV cartridge assembly 1′ can be desirable where the patient is unlikely to touch or move the roller wheel 40′.

Moreover, the roller wheel 40′ has a hub 44′ that is coupled to a hub slot 24′ of the tapered track 22′. The coupling between the hub 44′ and the hub slot 24′ reduces the likelihood of the roller wheel 44′ falling out of the tapered track 22′.

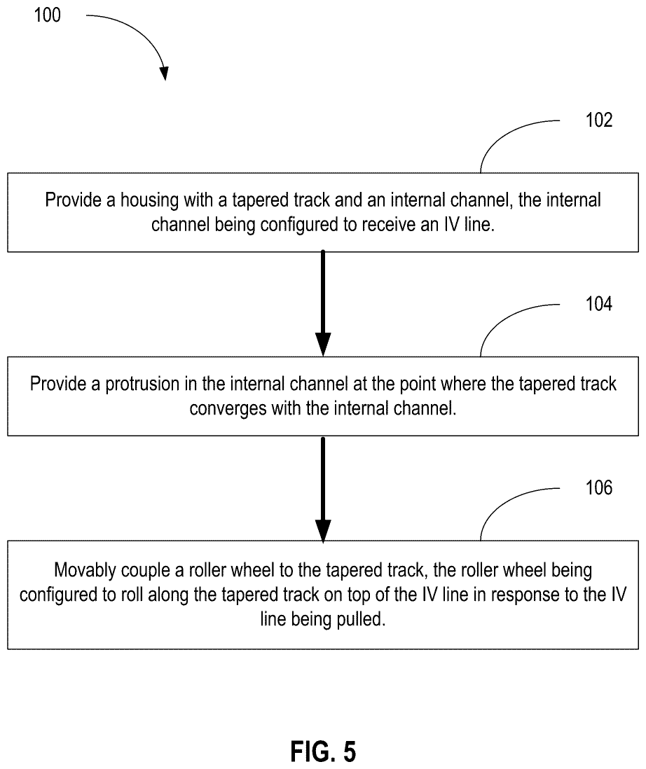

The present disclosure also includes methods of manufacturing the IV cartridge assemblies described herein. FIG. 5 depicts a flow chart of the method of manufacture of the IV cartridge assembly, according to various aspects of the subject technology. The method 100 can start at step 102 with providing a housing that has a tapered track and an internal channel. As described above, the internal channel is configured to receive an IV line from an IV set. The method 100 also includes providing a protrusion in the internal channel at step 104. The tapered track converges with the internal channel at the protrusion. Next, a roller wheel is coupled to the tapered track at step 106.

To manufacture the IV cartridge assembly depicted in FIGS. 1-3, providing the housing includes providing a top portion and a base portion and then moveably coupling the top portion to the base portion with a hinge. The top portion includes the tapered track, and the base portion includes the internal channel. When manufacturing this implementation of the IV cartridge assembly, the roller wheel is coupled to the top portion of the IV cartridge assembly via the tapered track. The top portion and the base portion can separate in an open position and come together in a closed position. The IV cartridge assembly can be locked when in the closed position.

As described above, the IV cartridge assembly produced by the method described herein is configured to be coupled to an IV set, a pressure sensor, and a processor.

Those of skill in the art would appreciate that the various illustrative blocks, modules, elements, components, methods, and algorithms described herein may be implemented in connection with electronic hardware, computer software, or combinations of both. To illustrate this interchangeability of hardware and software, various illustrative blocks, modules, elements, components, and methods have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. An intravenous (IV) cartridge assembly, the IV cartridge assembly comprising: an anchor configured to couple the IV cartridge assembly to a patient; an internal channel traversing the IV cartridge assembly, the internal channel being configured to receive an IV line; a protrusion formed in a bed of the internal channel; a tapered track within the IV cartridge assembly and above the internal channel; and a roller wheel configured to roll along the tapered track, wherein the roller wheel is configured to sit on top of the IV line without interfering with fluid flow through the IV line, wherein, in response to the IV line being pulled, the roller wheel is configured to roll towards the protrusion, wherein, when the roller wheel reaches the protrusion, the roller wheel is configured to pinch the IV line against the protrusion and occlude the IV line.

Clause 2. The IV cartridge assembly of Clause 1, wherein the tapered track converges with the internal channel at the protrusion.

Clause 3. The IV cartridge assembly of Clause 1, wherein the roller wheel is accessible from outside the IV cartridge assembly such that the roller wheel can be rolled away from the protrusion to reset the IV cartridge assembly and resume the fluid flow through the IV line.

Clause 4. The IV cartridge assembly of Clause 1, further comprising a hinge that couples a base portion of the IV cartridge assembly to a top portion of the IV cartridge assembly, wherein the base portion comprises the anchor, the internal channel, and the protrusion, wherein the top portion comprises the tapered track and conceals the roller wheel from a surrounding environment, wherein the top portion is rotatable relative to the base portion via the hinge such that the roller wheel is accessible and can be rolled away from the protrusion to reset the IV cartridge assembly and resume the fluid flow through the IV line.

Clause 5. The IV cartridge assembly of Clause 4, wherein the top portion is configured to be locked to the base portion such that the top portion is only rotatable relative to the base portion when the IV cartridge assembly is unlocked.

Clause 6. The IV cartridge assembly of Clause 1, wherein the IV cartridge assembly is configured to receive the IV line between the bed of the internal channel and the roller wheel.

Clause 7. The IV cartridge assembly of Clause 1, wherein the protrusion is configured to raise a portion of the IV line up into the tapered track.

Clause 8. The IV cartridge assembly of Clause 1, wherein the roller wheel comprises a disc with ridges on a circumference of the disc.

Clause 9. The IV cartridge assembly of Clause 8, wherein the ridges are configured to grip the IV line and facilitate the roller wheel rolling along the IV line when the IV line is pulled.

Clause 10. The IV cartridge assembly of Clause 1, wherein the IV cartridge assembly is configured to couple with a pressure sensor that measures pressure in the IV line to identify when the roller wheel reaches the protrusion and a processor that notifies caregivers that the IV line is occluded.

Clause 11. An intravenous (IV) cartridge assembly, the IV cartridge assembly comprising: an internal channel traversing the IV cartridge assembly, the internal channel being configured to receive an IV line; a protrusion formed in a bed of the internal channel; a tapered track within the IV cartridge assembly and adjacent to the internal channel; and a roller wheel positioned inside the tapered track and configured to roll along the tapered track above the IV line, wherein the roller wheel is configured to, in response to the IV line being pulled, roll on top of the IV line, without interfering with fluid flow through the IV line until the IV line is pulled beyond a threshold, wherein, when the IV line is pulled beyond the threshold, the roller wheel is configured to towards the protrusion, pinch the IV line against the protrusion, and occlude the IV line.

Clause 12. The IV cartridge assembly of Clause 11, wherein the tapered track converges with the internal channel at the protrusion.

Clause 13. The IV cartridge assembly of Clause 11, wherein the roller wheel is accessible from outside the IV cartridge assembly such that the roller wheel can be rolled away from the protrusion to reset the IV cartridge assembly and resume the fluid flow through the IV line.

Clause 14. The IV cartridge assembly of Clause 11, further comprising a hinge that couples a base portion of the IV cartridge assembly to a top portion of the IV cartridge assembly, wherein the base portion comprises the internal channel and the protrusion, wherein the top portion comprises the tapered track and conceals the roller wheel from a surrounding environment, wherein the top portion is rotatable relative to the base portion via the hinge such that the roller wheel is accessible and can be rolled away from the protrusion to reset the IV cartridge assembly and resume the fluid flow through the IV line.

Clause 15. The IV cartridge assembly of Clause 14, wherein the top portion is configured to be locked to the base portion such that the top portion is only rotatable relative to the base portion when the IV cartridge assembly is unlocked.

Clause 16. The IV cartridge assembly of Clause 11, wherein the IV cartridge assembly is configured to couple with a pressure sensor that measures pressure in the IV line to identify when the IV line is pulled beyond the threshold and a processor that notifies caregivers that the IV line is occluded.

Clause 17. A method for manufacturing an intravenous (IV) cartridge assembly, the method comprising: providing a housing, the housing comprising a tapered track and an internal channel, wherein the internal channel is configured to receive an IV line; providing a protrusion in the internal channel, the tapered track converging with the internal channel at the protrusion; and movably coupling a roller wheel to the tapered track, the roller wheel being configured to roll along the tapered track in response to the IV line being pulled, wherein the roller wheel is configured to roll along the IV line without interfering with fluid flow through the IV line until the roller wheel reaches the protrusion, at which point the roller wheel is configured to pinch the IV line against the protrusion and occlude the IV line.

Clause 18. The method of Clause 17, wherein providing the housing comprises providing a top portion of the housing comprising the tapered track, providing a base portion of the housing comprising the internal channel, and coupling the top portion to the base portion via a hinge such that the top portion is rotatable relative to the base portion via the hinge.

Clause 19. The method of Clause 18, further comprising locking the top portion to the base portion such that the top portion is only rotatable relative to the base portion when the IV cartridge assembly is unlocked.

Clause 20. The method of Clause 17, further comprising coupling a pressure sensor and a processor to the IV cartridge assembly, wherein the pressure sensor is configured to measure pressure in the IV line to identify when the roller wheel reaches the protrusion and the processor is configured to notify caregivers that the IV line is occluded.

Further Consideration:

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

What is claimed is:

1. An intravenous (IV) cartridge assembly, the IV cartridge assembly comprising:
an anchor configured to couple the IV cartridge assembly to a patient;
an internal channel traversing the IV cartridge assembly, the internal channel being configured to receive an IV line;
a protrusion formed in a bed of the internal channel;
a tapered track within the IV cartridge assembly and above the internal channel; and
a roller wheel configured to roll along the tapered track,
wherein the roller wheel is configured to sit on top of the IV line without interfering with fluid flow through the IV line,
wherein, in response to the IV line being pulled, the roller wheel is configured to roll towards the protrusion,
wherein, when the roller wheel reaches the protrusion, the roller wheel is configured to pinch the IV line against the protrusion and occlude the IV line.

2. The IV cartridge assembly of claim 1, wherein the tapered track converges with the internal channel at the protrusion.

3. The IV cartridge assembly of claim 1, wherein the roller wheel is accessible from outside the IV cartridge assembly such that the roller wheel can be rolled away from the protrusion to reset the IV cartridge assembly and resume the fluid flow through the IV line.

4. The IV cartridge assembly of claim 1, further comprising a hinge that couples a base portion of the IV cartridge assembly to a top portion of the IV cartridge assembly,
wherein the base portion comprises the anchor, the internal channel, and the protrusion,
wherein the top portion comprises the tapered track and conceals the roller wheel from a surrounding environment,
wherein the top portion is rotatable relative to the base portion via the hinge such that the roller wheel is accessible and can be rolled away from the protrusion to reset the IV cartridge assembly and resume the fluid flow through the IV line.

5. The IV cartridge assembly of claim 4, wherein the top portion is configured to be locked to the base portion such that the top portion is only rotatable relative to the base portion when the IV cartridge assembly is unlocked.

6. The IV cartridge assembly of claim 1, wherein the IV cartridge assembly is configured to receive the IV line between the bed of the internal channel and the roller wheel.

7. The IV cartridge assembly of claim 1, wherein the protrusion is configured to raise a portion of the IV line up into the tapered track.

8. The IV cartridge assembly of claim 1, wherein the roller wheel comprises a disc with ridges on a circumference of the disc.

9. The IV cartridge assembly of claim 8, wherein the ridges are configured to grip the IV line and facilitate the roller wheel rolling along the IV line when the IV line is pulled.

10. The IV cartridge assembly of claim 1, wherein the IV cartridge assembly is configured to couple with a pressure sensor that measures pressure in the IV line to identify when the roller wheel reaches the protrusion and a processor that notifies caregivers that the IV line is occluded.

11. An intravenous (IV) cartridge assembly, the IV cartridge assembly comprising:
an internal channel traversing the IV cartridge assembly, the internal channel being configured to receive an IV line;
a protrusion formed in a bed of the internal channel;
a tapered track within the IV cartridge assembly and adjacent to the internal channel; and
a roller wheel positioned inside the tapered track and configured to roll along the tapered track above the IV line,
wherein the roller wheel is configured to, in response to the IV line being pulled, roll on top of the IV line, without interfering with fluid flow through the IV line until the IV line is pulled beyond a threshold,
wherein, when the IV line is pulled beyond the threshold, the roller wheel is configured to towards the protrusion, pinch the IV line against the protrusion, and occlude the IV line.

12. The IV cartridge assembly of claim 11, wherein the tapered track converges with the internal channel at the protrusion.

13. The IV cartridge assembly of claim 11, wherein the roller wheel is accessible from outside the IV cartridge assembly such that the roller wheel can be rolled away from the protrusion to reset the IV cartridge assembly and resume the fluid flow through the IV line.

14. The IV cartridge assembly of claim 11, further comprising a hinge that couples a base portion of the IV cartridge assembly to a top portion of the IV cartridge assembly,
wherein the base portion comprises the internal channel and the protrusion,
wherein the top portion comprises the tapered track and conceals the roller wheel from a surrounding environment,
wherein the top portion is rotatable relative to the base portion via the hinge such that the roller wheel is accessible and can be rolled away from the protrusion to reset the IV cartridge assembly and resume the fluid flow through the IV line.

15. The IV cartridge assembly of claim 14, wherein the top portion is configured to be locked to the base portion such that the top portion is only rotatable relative to the base portion when the IV cartridge assembly is unlocked.

16. The IV cartridge assembly of claim 11, wherein the IV cartridge assembly is configured to couple with a pressure sensor that measures pressure in the IV line to identify when the IV line is pulled beyond the threshold and a processor that notifies caregivers that the IV line is occluded.

17. A method for manufacturing an intravenous (IV) cartridge assembly, the method comprising:
providing a housing, the housing comprising a tapered track and an internal channel, wherein the internal channel is configured to receive an IV line;
providing a protrusion in the internal channel, the tapered track converging with the internal channel at the protrusion; and
movably coupling a roller wheel to the tapered track, the roller wheel being configured to roll along the tapered track in response to the IV line being pulled,
wherein the roller wheel is configured to roll along the IV line without interfering with fluid flow through the IV line until the roller wheel reaches the protrusion, at which point the roller wheel is configured to pinch the IV line against the protrusion and occlude the IV line.

18. The method of claim 17, wherein providing the housing comprises providing a top portion of the housing comprising the tapered track, providing a base portion of the housing comprising the internal channel, and coupling the top portion to the base portion via a hinge such that the top portion is rotatable relative to the base portion via the hinge.

19. The method of claim 18, further comprising locking the top portion to the base portion such that the top portion is only rotatable relative to the base portion when the IV cartridge assembly is unlocked.

20. The method of claim 17, further comprising coupling a pressure sensor and a processor to the IV cartridge assembly, wherein the pressure sensor is configured to measure pressure in the IV line to identify when the roller wheel reaches the protrusion and the processor is configured to notify caregivers that the IV line is occluded.
